(19) United States
(12) Patent Application Publication
(10) Pub. No.: US 2024/0254164 A1
(43) Pub. Date: Aug. 1, 2024

(54) PURIFICATION OF SPECIFIC TRIPEPTIDE BY ACIDIFICATION

(71) Applicant: DSM IP Assets B.V., Heerlen (NL)

(72) Inventors: Marc HEIDL, Kaiseraugst (CH); Thomas SCHAERER, Kaiseraugst (CH); Alexander SCHLIFKE-POSCHALKO, Kaiseraugst (CH)

(21) Appl. No.: 18/559,251

(22) PCT Filed: May 3, 2022
(86) PCT No.: PCT/EP2022/061790

§ 371 Date: Nov. 6, 2023

(30) Foreign Application Priority Data
May 10, 2021 (EP) 21172935.5

(51) Int. Cl. C07K 5/083; A61K 8/64; A61K 38/00; A61Q 19/08

(52) U.S. Cl. CPC C07K 5/081; A61K 8/64; A61Q 19/08; A61K 38/00; A61K 2800/805

(57) ABSTRACT

The present invention relates to purification of a tripeptide of formula (I). By using said method, the tripeptide of formula (I) is obtained in a very pure form and due to this effect the purified tripeptide can be used in a variety of cosmetic and pharmaceutic compositions which are not accessible for the respective unpurified tripeptide.

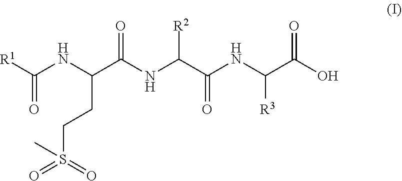

### TECHNICAL FIELD

The present invention relates to the purification of a tripeptide of the formula (I) and its use in cosmetic and pharmaceutical compositions.

### BACKGROUND OF THE INVENTION

Peptides, in general, play an important role in skin care in particular as anti-aging (anti-wrinkle, skin elasticity, contour remodeling) agents. However, many peptides are susceptible to oxidation and thus not stable when incorporated into a cosmetic composition.

The tripeptide of formula (I), as shown later-on in this document, is particularly well suited for cosmetic and pharmaceutical compositions.

This tripeptide of formula (I) (Ac-Met(O2)-Val-Val-OH) shows significant lower susceptibility against oxidation by atmospheric oxygen as compared to the corresponding tripeptide of the formula (I0) (Ac-Met-Val-Val-OH), as shown later-on in this document.

It has been shown that the tripeptide of formula (I) has an intense colour which severely limits its application in the field of cosmetics and pharmaceuticals. It has been found that this off-colour are due to impurities which are very difficult to remove by simple purification methods.

Attempts to find a suitable easy and economic method of purification have failed so far.

### SUMMARY OF THE INVENTION

Therefore, the problem to be solved by the present invention is to offer an economically favourable and efficient method of purification of compound of the formula (I).

Surprisingly, it has been found that the method of claim 1 offers a solution to this process. An essential feature of this novel and inventive method is the use of very low pH in the purification.

It has been found that said process yield the tripeptide of the formula (I) without off-colour, i.e. that the tripeptide is white, in other words, colourless or at least essentially colourless, which is crucial for many applications, in such a degree as not yet known or expected.

It has been particularly found that there exist particularly two preferred embodiments of this method which offers the above mentioned advantages.

Due to this very efficient purification method, the tripeptide of the formula (I) can now be used also in cosmetic and pharmaceutic compositions which have been blocked up to now because the off-colour of tripeptide available until now.

Further aspects of the invention are subject of further independent claims. Particularly preferred embodiments are subject of dependent claims.

### DETAILED DESCRIPTION OF THE INVENTION

In a first aspect the present invention relates to a method of purification a tripeptide of the formula (I)

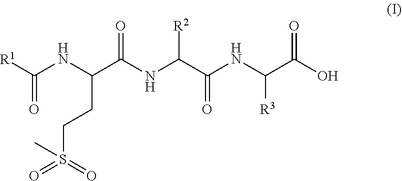

comprising the steps
a) providing a composition comprising a tripeptide of the formula (I)
b) forming an aqueous solution of the composition of step a)
c) bringing the composition of step b) to a pH≤2.5;
d) forming a precipitation of the tripeptide of the formula (I);
e) separating the precipitated tripeptide of the formula (I) formed in step d)
wherein
R¹ represents either H or a C₁₋₁₆-alkyl group or an aryl group or a C₇₋₁₆-aralkyl group or a C₇₋₁₆-alkylaryl group, which are optionally substituted by up to three hydroxy groups; and
R² and R³
   either represent independently from each other a C₁₋₁₀-alkyl group, which are optionally substituted by up to three hydroxy groups;
   or represent together a C₂₋₁₀-alkylene group, which is optionally substituted by up to three hydroxy groups.

For sake of clarity, some terms used in the present document are defined as follows:

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂—CH₃ is considered as a C₄-alkyl group.

Analogously, a “C_{x-y}-alkylene” group is in the present document an alkylene group comprising x to y carbon atoms, i.e., for example, a C₂₋₃-alkylene group is an alkylene group comprising 2 to 3 carbon atoms. The alkylene group can be linear or branched. For example, —CH₂—CH₂—CH₂— and —CH(CH₃)—CH₂— and —C(CH₂—CH₃)— and —C(CH₃)₂— are all considered as a C₃-alkylene group.

An “aryl group” is an aromatic substituent. Preferred aryl groups are phenyl or naphthyl groups.

An “aralkyl” group is an alkyl group which is substituted by an aryl group.

Accordingly, in the present document, a “C_{x-y}-aralkyl” group is an aralkyl group comprising x to y carbon atoms, i.e., for example, a C₇₋₁₆-aralkyl group is an aralkyl group comprising 7 to 16 carbon atoms. The aralkyl group can be linear or branched. For example, benzyl group (—CH₂—C₆H₅) is considered as a C₇-aralkyl group.

An “alkylaryl” group is an aryl group which is substituted by an alkyl group.

Accordingly, in the present document, a “C_{x-y}-alkylaryl” group is an alkylaryl group comprising x to y carbon atoms, i.e., for example, a C₇₋₁₆-alkylaryl group is an alkylaryl group comprising 7 to 16 carbon atoms. The alkylaryl group can be linear or branched. For example, the tolyl group (—C₆H₄CH₃) is considered as a C₇-alkylaryl group and the xylyl group (—C₆H₃(CH₃)₂) is considered as a C₈-alkylaryl group.

In case identical labels for symbols or groups are present in several formulae, in the present document, the definition of said group or symbol made in the context of one specific formula applies also to other formulae which comprises the same said label.

The term “independently from each other” in this document means, in the context of substituents, moieties, or groups, that identically designated substituents, moieties, or groups can occur simultaneously with a different meaning in the same molecule.

In the present document, any dotted line in formulae represents the bond by which a substituent is bound to the rest of a molecule.

In the present document any bond having dotted line

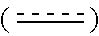

in a chemical formula represents independently from each other either a single carbon-carbon bond or a double carbon-carbon bond.

Any wavy line in any formula of in this document represents a carbon-carbon bond and which when linked to the carbon-carbon double bond is either in the Z or in the E-configuration. It is preferred in all molecules that the carbon-carbon double bond is in the E-configuration.

The “pKₐ” is commonly known as negative decadic logarithm of the acid dissociation constant (pKₐ=−log₁₀ Kₐ). The pKₐ are measured at standard temperature and pressure.

Ambient temperature in the present document means a temperature of 23° C.-25° C., preferably 23° C.

A “precipitation” as used in this document is a solid which is formed from a solution and separates from the liquid phase by the influence of gravity of earth, within a time of maximum 1 hours. Therefore, solids such as sols, gels or colloid dispersions, are not considered as precipitated in the sense of this document.

In step a) of the said process a composition comprising a tripeptide of the formula (I) is provided.

Typically said composition is a crude product of the tripeptide of the formula (I) and more preferably the composition comprises water. The tripeptide is preferably prepared either by solid phase peptide synthesis using the respective FMOC-protected amino acids or by liquid phase peptide synthesis using the respective BOC-protected amino acids to form the tripeptide of the formula (I0), followed by an oxidation of the S—CH₃ group to the SO₂—CH₃ group by a suitable oxidizing agent.

The oxidation of the tripeptide of the formula (I0) to prepare the tripeptide of the formula (I) is particularly performed by hydrogen peroxide. Typically a stoichiometric excess of the oxidizing agent is used to assure quantitative formation of the tripeptide of the formula (I). The excess of oxidizing agent is preferably quenched, such as by using dimethyl sulfoxide (DMSO).

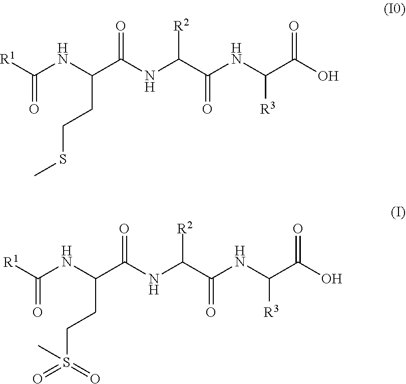

R¹ represents in one embodiment the tripeptide of the formula (I) a C₁₋₁₆-alkyl group which is optionally substituted by up to three hydroxy groups. R² and R³ represents in one of the embodiments a C₁₋₁₀-alkyl group which is optionally substituted by up to three hydroxy groups.

Preferably, the C₁₋₁₆-alkyl group or the C₁₋₁₀-alkyl group, which is optionally substituted by up to three hydroxy groups, is selected from the group consisting of methyl, ethyl, n-propyl, 1-methylethyl, 3-hydroxypropyl, 2,3-dihydroxypropyl, 3-hydroxybutyl, 4-hydroxybutyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, 3,5,5-trimethylhexyl and 2,3-dihydroxypropyl groups, preferably methyl, ethyl, propyl, butyl, hexyl, heptyl, octyl or 2,3-hydroxypropyl group.

In another embodiment, R² and R³ represent together a C₂₋₁₀-alkylene group, which is optionally substituted by up to three hydroxy groups. Preferably, the C₂₋₁₀-alkylene group, is selected from the group consisting of ethylene, propylene, butylene, pentylene and hexylene group.

Most preferably, the C₁₋₁₆-alkyl group and/or the C₁₋₁₀-alkyl group and/or the aryl group and/or the C₇₋₁₆-aralkyl group and or the C₇₋₁₆-alkylaryl group and/or the C₂₋₁₀-alkylene group are not substituted by any hydroxy groups.

It is preferred that in formula (I) R¹ represents a methyl group or H, and that R² and R³ represent both isopropyl groups.

It is most preferred that the compound of the formula (I) is the compound of the formula (I-A)

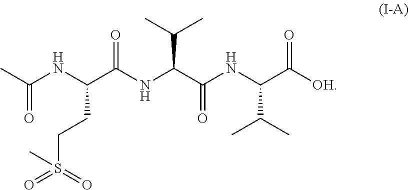 [SMILES: CC(=O)N[C@@H](CCS(C)(=O)=O)C(=O)N[C@H](C(=O)N[C@H](C(=O)O)C(C)C)C(C)C]

In step b) an aqueous solution of the composition of the step a) is formed.

It is preferred that solution is prepared to assure that the concentration is as high as possible in formula (I).

It has been shown, that particularly two preferred embodiments of the described process are particularly effective and cost-advantageous:

In one these preferred embodiments, in step b) a sufficient amount of water is added to the prepare a solution of a pH>3. It is preferred that in this embodiment water is added in combination with a base. Said base is preferably a water-soluble base, particularly an inorganic base, preferably an inorganic base selected from the group consisting of carbonates, bicarbonates, oxides, hydroxides and oxyhydroxides, preferably of metal ions, particularly of alkali metals ions. A preferred base is LiOH.

In this embodiment, it is preferred that step b) is performed at a temperature of between 10° C. and 30° C., preferably at ambient temperature.

In this embodiment, step c), i.e. bringing the composition of step b) to a pH≤2.5, preferably to a pH≤2, is performed after step b) by preferably adding an acid to the solution of step b). As a consequence of bringing the pH to a pH s 2.5, a precipitation of the tripeptide of the formula (I) is formed when the temperature is at a temperature of below 30° C., preferably between 30° C. and 1° C., more preferably between 25° C. and 4° C.

In another preferred embodiment, the composition of step a) is present as in an acidified form. In step b) water is added and heated to an elevated temperature, preferably to a temperature of more than 80° C., preferably to a temperature of between 80° C. and 100° C., more preferably to reflux temperature to form a solution at this temperature.

Depending on the degree of acidification of the composition it may be necessary to add acid to obtain a pH≤2.5, preferably to a pH≤2, in step d) at the above elevated temperature preferably to a temperature of more than 80° C., preferably to 90° C.-100° C., more preferably to reflux temperature.

Upon cooling of the composition of step d) of this embodiment to a temperature of between 30° C. and 0° C., preferably of between 25° C. and 0° C., a precipitation of the tripeptide of the formula (I) is formed in step d).

It is preferred that the pH in step b) is brought to a pH of ≤2.5, preferably of ≤2, by addition of a strong acid, preferably by HCl.

It is has been shown that if the pH in step c) is higher than 2.5, no precipitation is formed or that that the purification is significantly lower than obtained by the process of invention.

In step d) a precipitation is formed. It is important to stress that this precipitation separates from the liquid phase by the influence of gravity of earth, within a time of maximum 1 hours. It is preferred that the precipitation is in the form of particles of a mean diameter of more than 1 micrometre, preferably more than 10 micrometre. It is preferred that the mean diameter of said particles are in the range of between 1 μm and 100 μm, particularly between 5 μm and 50 μm, more particularly between 5 μm and 30 μm. The precipitation can be an amorphous or crystalline form. Preferably, the precipitated tripeptide of the formula (I) is in crystalline form.

In step e) the precipitated tripeptide of the formula (I) formed in step d) is separated. The separation is preferably a filtration over a Nutsche filter or a Büchner funnel or an agitated nutsche filter (ANF) or a glass frit (sintered glass) filter. Preferably, for this separation step e) a vacuum is applied for the filtration.

Preferably, for this separation step e) a vacuum is applied for the filtration.

If needed or desired, the tripeptide of the formula (I), separated in step e) can be subjected to one or more additional purification cycles comprising steps a) to e).

Typically, additional purification cycles are not needed as the process provides the tripeptide of the formula (I) in a very pure form after the (first) step e).

It has been found that the above process is able to provide the tripeptide of the formula (I) in an extraordinary highly pure form.

The so purified tripeptide of the formula (I) is particularly advantageous as it has no off-colour, i.e. that the tripeptide is white, in other words, colourless or at least essentially colourless, which is crucial for many applications of the tripeptide.

In a very preferred application, the tripeptide of the formula (I), purified by the process as described above is its use in cosmetics or pharmaceuticals and their manufacturing.

Hence, in a further aspect, the present invention relates to a cosmetic or pharmaceutical composition comprising a tripeptide of the formula (I) which has been purified by a method as described above in great details.

The term ‘cosmetic composition’ refers to compositions which are used to treat, care for or improve the appearance of the skin and/or the scalp. Particular advantageous cosmetic compositions according to the present invention are skin care compositions.

The cosmetic or pharmaceutical compositions according to the invention are preferably intended for topical application, which is to be understood as the external application to keratinous substances, such as in particular the skin.

The term ‘cosmetically acceptable carrier’ as used herein refers to a physiologically acceptable medium which is compatible with keratinous substances. Suitable carriers are well known in the art and are selected based on the end-use application. Preferably, the carriers of the present invention are suitable for application to skin (e.g., sunscreens, creams, milks, lotions, masks, serums, hydrodispersions, foundations, creams, creamgels, or gels etc.). Such carriers are well-known to one of ordinary skill in the art, and can include one or more compatible liquid or solid filler, diluent, excipient, additive or vehicle which are suitable for application to skin. Particularly preferred cosmetically acceptable carrier is selected from the group consisting of water, oils, fats, waxes, organic solvents and fillers. The exact amount of carrier will depend upon the level of the compound of formula (I) and any other optional ingredients that one of ordinary skill in the art would classify as distinct from the carrier (e.g., other active components). The compositions of the present invention preferably comprise from about 75% to about 99.999%, more preferably from about 85% to about 99.99%, still more preferably from 90% to about 99%, and most preferably, from about 93% to about 98%, by weight of the composition, of a carrier.

The cosmetic or pharmaceutical compositions of the present invention can be formulated into a wide variety of product types, including creams, waxes, pastes, lotions, milks, mousses, gels, oils, tonics, and sprays. Preferably the compounds of formula (I) are formulated into lotions, creams, gels, and tonics. These product forms may be used for a number of applications, including, but not limited to, hand and body lotions, facial moisturizers, anti-ageing preparations, make-ups including foundations, and the like. Any additional components required to formulate such products vary with product type and can be routinely chosen by a person skilled in the art.

If compositions of the present invention are formulated as an aerosol and applied to the skin as a spray-on product, a propellant is added to the composition.

The cosmetic or pharmaceutical compositions according to the present invention can be prepared by conventional methods in the art such as e.g. by admixing a compound of formula (I) with all the definitions and preferences given herein with the cosmetically acceptable carrier. The cosmetic compositions of the invention (including the carrier) may comprise further conventional cosmetic adjuvants and additives, such as preservatives/antioxidants, fatty substances/oils, water, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the cosmetic or pharmaceutical compositions according to the invention may also comprise further cosmetically active ingredients conventionally used in cosmetic compositions. Exemplary active ingredients encompass further self-tanning agents, UV-filters, agents for the treatment of hyperpigmentation; agents for the prevention or reduction of inflammation; firming, moisturizing, soothing, and/or energizing agents as well as agents to improve elasticity and skin barrier.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of the active ingredients as well as the cosmetic excipients, diluents, adjuvants, additives etc. can, based on the desired product form and application, easily be determined by the person skilled in the art. The additional ingredients can either be added to the oily phase, the aqueous phase or separately as deemed appropriate.

The cosmetically active ingredients useful herein can in some instances provide more than one benefit or operate via more than one mode of action.

Of course, a person skilled in the art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

These compositions can be used particularly for the prevention, treatment and/or reduction of wrinkles, improvement of skin elasticity and/or for contour remodeling. The compositions are particularly brought in contact to a skin in need of such a treatment with a compound of formula (I) with all the definitions and preferences as given herein and optionally appreciating the effect.

Therefore, the invention relates, in a further aspect, to a process of manufacturing a cosmetic or pharmaceutical composition comprising the steps d
i) purifying a tripeptide of the formula (I) by a method as described above in great detail yielding a purified tripeptide of the formula (I);
ii) providing a cosmetically acceptable carrier
iii) combining the purified tripeptide of the formula (I) of step i) with the cosmetically acceptable carrier of step ii) and with optionally further ingredients to yield a cosmetic or pharmaceutical composition.

Accordingly, as described above, said process of manufacturing yields a cosmetic or pharmaceutical composition. Therefore, a further aspect of the present invention relates to a cosmetic or pharmaceutical composition, particularly a cosmetic composition, which is produced according to a said process.

As shown above, the tripeptide of the formula (I), purified by the process as described above can be used in a cometic composition.

Hence, in a further aspect, the present invention relates to a use of a tripeptide of the formula (I), which is purified using a method as described above in great details in a cometic composition.

### EXAMPLES

The present invention is further illustrated by the following experiments.

### Synthesis of Tripeptides

The following tripeptides have been prepared by solid phase peptide synthesis using FMOC-protected amino acids from methionine (Met), valine (Val), serine (Ser), proline (Pro), alanine (Ala) and glutamine (Gln):

| | |
|---|---|
| Ac-Met-Val- Val-OH | 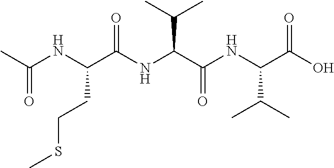 [SMILES: CSCC[C@H](NC(C)=O)C(=O)N[C@H](C(=O)N[C@H](C(=O)O)C(C)C)C(C)C] |
| Ac-Ser-Pro- Ala-OH | 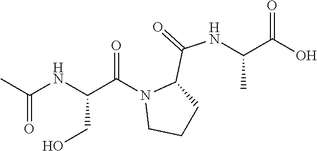 [SMILES: CC(=O)N[C@@H](CO)C(=O)N1CCC[C@H]1C(=O)N[C@@H](C)C(=O)O] |
| Ac-Gln-Pro- Ala-OH | 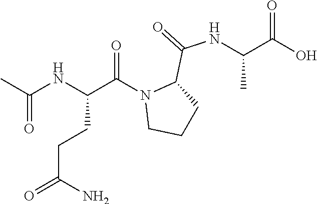 [SMILES: CC(=O)N[C@@H](CCC(N)=O)C(=O)N1CCC[C@H]1C(=O)N[C@@H](C)C(=O)O] |

The tripeptide Ac-Met-Val-Val-OH has been treated with H₂O₂ an stochiometric excess followed by quenching with DMSO to yield the tripeptide of the formula (I-A) (Ac-Met(O2)-Val-Val-OH) which has been separated and used for the following examples

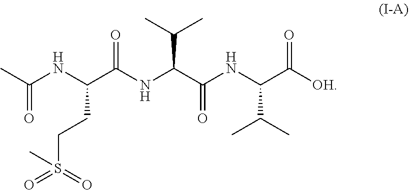 [SMILES: CC(=O)N[C@@H](CCS(C)(=O)=O)C(=O)N[C@H](C(=O)N[C@H](C(=O)O)C(C)C)C(C)C]

### First Series of Examples

54 mg Ac-Met(O2)-Val-Val-OH have been dissolved in 0.2 ml 0.1 M aqueous LiOH. A clear solution is formed. The pH was lowered from 9 to the pH as indicated in table 1 by adding 2M HCl and cooled to a temperature of 4° C.

TABLE 1
Different pH.

| Ex. | pH | Observation after cooling |
|---|---|---|
| 1 | 2 | Fine particles formed |
| Ref.1 | 3 | Opaque solution. No particles formed |

In example 1, a precipitation the tripeptide in fine particles at the bottom of the vessel was observed is formed which was separated by filtration over a glass frit filter. In the comparative example Ref.1 the sample became slightly opaque while cooling to 4° C., but no particles have been formed and, hence, no solid tripeptide could be separated or isolated.

### Example 2

67.3 g acidified Ac-Met(O2)-Val-Val-OH was dissolved in 500 ml water (pH 1.9 of solution) at reflux. The solution was cooled to 4° C. within 2.5 hours and kept at 4° C. during 30 minutes. The fine particles formed at the bottom of the vessel have been collected by a Büchner funnel.

### Second Series of Examples

Aqueous solutions of different tripeptides acidified to the pH as given in table 2 were heated to reflux temperature. The clear solutions (0.5 mol/L) have been cooled to room temperature.

TABLE 2
Different tripeptides

| Ex. | Tripeptide | pH | Observation after cooling |
|---|---|---|---|
| 3 | Ac-Met(O2)-Val-Val-OH | 2 | Fine particles formed |
| Ref.2 | Ac-Ser-Pro-Ala-OH | 0.9 | No particles formed |
| Ref.3 | Ac-Gln-Pro-Ala-OH | 1.5 | No particles formed |

The fine particles of example 3 formed at the bottom of the vessel have been collected by a filtration over a glass frit filter. Hence, in case of examples Ref.2 and Ref.3, no solid tripeptide could be separated or isolated.

1. A method of purification a tripeptide of the formula (I) 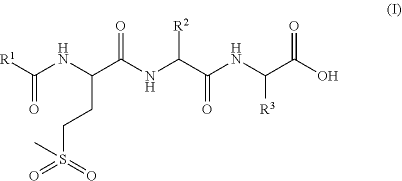
comprising the steps
a) providing a composition comprising a tripeptide of the formula (I)
b) forming an aqueous solution of the composition of step a)
c) bringing the composition of step b) to a pH≤2.5;
d) forming a precipitation of the tripeptide of the formula (I);
e) separating the precipitated tripeptide of the formula (I) formed in step d)
wherein
R¹ represents either H or a C₁₋₁₆-alkyl group or an aryl group or a C₇₋₁₆-aralkyl group or a C₇₋₁₆-alkylaryl group, which are optionally substituted by up to three hydroxy groups; and
R² and R³
either represent independently from each other a C₁₋₁₀-alkyl group, which are optionally substituted by up to three hydroxy groups;
or represent together a C₂₋₁₀-alkylene group, which is optionally substituted by up to three hydroxy groups.

2. The method according to claim 1, wherein
R¹ is methyl or H,
R² and R³ are both isopropyl.

3. The method according to claim 1, wherein the compound of the formula (I) is the compound of the formula (I-A)

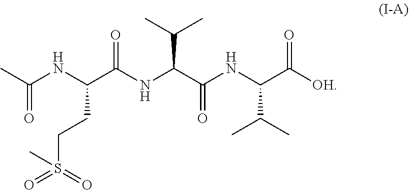 [SMILES: CC(=O)N[C@@H](CCS(C)(=O)=O)C(=O)N[C@H](C(=O)N[C@H](C(=O)O)C(C)C)C(C)C]

4. The method according to claim 1, wherein in step b) a sufficient amount of water, preferably in combination with a base, to the prepare a solution of a pH>3.

5. The method according to claim 4, wherein step b) is performed at a temperature of between 10° C. and 30° C., preferably at ambient temperature.

6. The method of claim 5, wherein in step d) acid is added to a pH≤2.5, preferably to a pH≤2, preferably at a temperature at a temperature of below 30° C., preferably between 30° C. and 1° C.

7. The method according to a claim 1, wherein composition of step a) is present as in an acidified form and that in step b) water is added and heated to an elevated temperature, preferably to a temperature of more than 80° C., preferably to a temperature of between 80° C. and 100° C., more preferably to reflux temperature to form a solution at this temperature.

8. The method according to claim 7, wherein step d) is performed by cooling the solution from the elevated temperature to a temperature of between 30° C. and 0° C., preferably of between 25° C. and 0° C.

9. The method according to claim 1, wherein in step b) the composition is brought to a pH of ≤2.5, preferably of ≤2, by addition of a strong acid, preferably by HCl.

10. The method according to claim 1, wherein the tripeptide of the formula (I) is prepared from the tripeptide of the formula (I0) by oxidation, particularly by oxidation with H₂O₂,

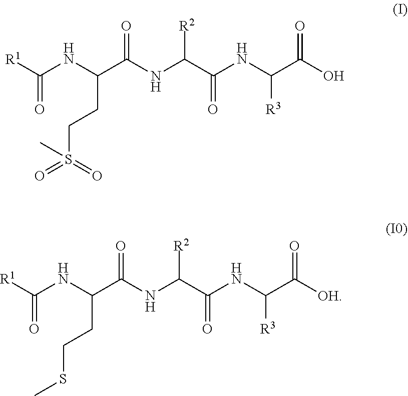

11. A process of manufacturing a cosmetic or pharmaceutical composition comprising the steps
i) purifying a tripeptide of the formula (I) by a method according to claim 1, yielding a purified tripeptide of the formula (I);
ii) providing a cosmetically acceptable carrier
iii) combining the purified tripeptide of the formula (I) of step i) with the cosmetically acceptable carrier of step ii) and with optionally further ingredients to yield a cosmetic or pharmaceutical composition.

12. A cosmetic or pharmaceutical composition which is produced according to a process according to claim 11.

13. A cosmetic or pharmaceutical composition comprising a tripeptide of the formula (I)
which has been purified by a method according to claim 1, and a cosmetically acceptable carrier 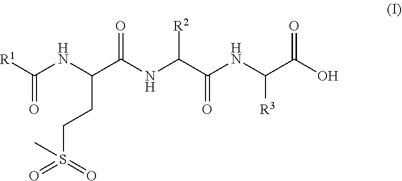
wherein
R¹ represents either H or a C₁₋₁₆-alkyl group or an aryl group or a C₇₋₁₆-aralkyl group or a C₇₋₁₆-alkylaryl group, which are optionally substituted by up to three hydroxy groups; and
R² and R³
either represent independently from each other a C₁₋₁₀-alkyl group, which are optionally substituted by up to three hydroxy groups;
or represent together a C₂₋₁₀-alkylene group, which is optionally substituted by up to three hydroxy groups.

14. Use of a tripeptide of the formula (I), which is purified using a method according to claim 1, in a cometic composition 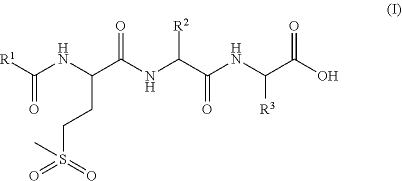
wherein
R¹ represents either H or a C₁₋₁₆-alkyl group or an aryl group or a C₇₋₁₆-aralkyl group or a C₇₋₁₆-alkylaryl group, which are optionally substituted by up to three hydroxy groups; and
R² and R³
either represent independently from each other a C₁₋₁₀-alkyl group, which are optionally substituted by up to three hydroxy groups;
or represent together a C₂₋₁₀-alkylene group, which is optionally substituted by up to three hydroxy groups.
